(19) United States
(12) Patent Application Publication
(10) Pub. No.: US 2025/0224395 A1
(43) Pub. Date: Jul. 10, 2025

(54) METHODS OF SOLUTION-PHASE KINETIC ANALYSIS ON A DIGITAL MICROFLUIDIC (DMF) DEVICE

(71) Applicant: Nicoya Lifesciences Inc., Kitchener (CA)

(72) Inventors: Gordon H. HALL, Cambridge (CA); Ryan Cameron DENOMME, Kitchener (CA); Michael PIAZZA, Etobicoke (CA)

(21) Appl. No.: 19/015,473

(22) Filed: Jan. 9, 2025

Related U.S. Application Data
(60) Provisional application No. 63/619,501, filed on Jan. 10, 2024.

(51) Int. Cl. G01N 33/557; G01N 33/543; G01N 33/96

(52) U.S. Cl. CPC G01N 33/557; G01N 33/54373; G01N 33/96

(57) ABSTRACT

Methods of solution-phase kinetic analysis on a digital microfluidic (DMF) device are disclosed. In some embodiments, the methods of solution-phase kinetic analysis on a DMF device provide a DMF system including a DMF device (or cartridge) further including one or more electrode arrangements. The methods may utilize a variety of detection methods including surface plasmon resonance and fluorescence based techniques.

### CROSS-REFERENCE

This application claims the benefit of priority to U.S. Provisional Application No. 63/619,501, filed Jan. 10, 2024, which is hereby incorporated by reference in its entirety herein.

### TECHNICAL FIELD

The subject matter relates generally to the detection of molecules, such as DNA, proteins, and the like, and more particularly to methods for determining solution phase kinetics for molecular interactions.

### BACKGROUND

Solution phase measurements (e.g., KinExA technology, MicroScale Thermophoresis (MST), Isothermal Titration calorimetry (ITC)) are typically used as confirmatory measurements for surface immobilization based techniques. Briefly, this consists of mixing the binding pair together in solution where one half of the pair is held at a constant concentration (A) and the other half varies (B). Next, the amount of free A is assessed for each concentration sample and the analyte affinity value K_{D} is measured.

This method may have certain advantages. In one example, this technique provides accurate affinity measurements as it is done in solution with no labels. This means any surface effect (charges, sterics, etc. . . . ) does not affect the affinity measurement. In another example, using this technique, a particularly wide dynamic range of affinities can be measured. This is especially true at the high K_{D} levels that are difficult to measure with other methods.

At the same time, this method may have certain disadvantages. In one example, using this method the experimental throughput is slow. For example, the time for the solution to come to equilibrium can be on the order of days. In another example, using this method it may be difficult to determine a reaction run time. For example, if the reaction time is too short then sample and data may be wasted. By contrast, if the reaction time is too long there is risk of sample degradation and time waste.

### SUMMARY

In an aspect, the present disclosure provides a method for determining solution phase properties of a binding complex. In some embodiments, the method may include the steps of: providing a first fluid comprising a known concentration of an analyte; contacting the first fluid with a sensor comprising an immobilized binding partner, wherein the immobilized binding partner is bound to a surface of the sensor and is configured to bind the analyte to form an immobilized binding complex; measuring a signal generated by the sensor in response to formation of the immobilized binding complex; generating a calibration curve using the signal generated by sensor; providing a second fluid comprising a known concentration of the analyte and a known concentration of free binding partner, wherein the free binding partner is configured to bind the analyte to form a free binding complex; contacting the second fluid with the sensor comprising the immobilized binding partner thereby forming a second immobilized binding complex; measuring a second signal generated by the sensor in response to formation of the second immobilized binding complex; and comparing the second signal to the calibration curve to determine one or more properties of the free binding complex.

In some embodiments, the analyte, the immobilized binding partner, and the free binding partner are biomolecules. In some embodiments, the biomolecules are selected from the group consisting of: antibodies, antibody fragments, recombinant proteins, oligonucleotides, lipids, small molecules, viruses and virus like particles (VLP), and whole cells.

In some embodiments, the immobilized binding partner and free binding partner are the same or substantially the same. In some embodiments, the immobilized binding complex and free binding complex are the same or substantially the same.

In some embodiments, the method may further include the steps of: partitioning the second fluid into one or more portions; contacting a portion of the one or more portions with the sensor comprising the immobilized binding partner thereby forming an additional immobilized binding complex; measuring one or more additional signals generated by the sensor in response to formation of the additional immobilized binding complex; and comparing the one or more additional signals to the calibration curve to determine one or more properties of the free binding complex.

In some embodiments, the one or more properties of the free binding complex is one or more of a concentration of analyte, on-rate, off-rate, or binding affinity.

In some embodiments, the method may further include the steps of: determining a sampling rate using the one or more properties of the free binding complex.

In some embodiments, any one of the first fluid, second fluid, and/or one or more portions are fluid droplets. In some embodiments, the fluid droplets are manipulated using digital microfluidics (DMF) mediated droplet operations.

In some embodiments, the sensor is a SPR or LSPR sensor. In some embodiments, the SPR or LSPR sensor comprises a surface having a nanostructured portion. In some embodiments, the first binding partner is immobilized to the nanostructured portion.

In some embodiments, the sensor is disposed within a gap of a microfluidic cartridge.

In some embodiments, the method may further include the step of: providing one or more signal enhancers configured to bind to the immobilized binding complex thereby enhancing the signal generated by the sensor. In some embodiments, the method may further include the step of: providing one or more signal enhancers configured to bind to the second immobilized binding complex thereby enhancing the second signal generated by the sensor. In some embodiments, the method may further include the step of: providing one or more signal enhancers configured to bind to the additional immobilized binding complexes thereby enhancing the one or more additional signals generated by the sensor.

In another aspect, the present disclosure provides a method for determining solution phase binding kinetics. In some embodiments, the method may include the steps of: providing a first fluid comprising a known concentration of an analyte and a known concentration of a free binding partner, wherein the free binding partner is configured to bind the analyte to form a free binding complex; contacting the first fluid with one or more magnetic beads functionalized with an immobilized binding partner, wherein the immobilized binding partner is configured to bind the analyte to form an immobilized binding complex; isolating the one or more magnetic beads; contacting the one or more magnetic beads with a second fluid comprising one or more types of labels, wherein the one or more types of labels are configured to bind the immobilized binding complex thereby forming a labeled binding complex; and measuring a signal generated by the labeled binding complex to determine one or more properties of the free binding complex.

In some embodiments, the analyte, the immobilized binding partner, and the free binding partner are biomolecules. In some embodiments, the biomolecules are selected from the group consisting of: antibodies, antibody fragments, recombinant proteins, oligonucleotides, lipids, small molecules, viruses and virus like particles (VLP), and whole cells.

In some embodiments, the one or more types of labels comprises fluorescent labels. In some embodiments, the one or more types of labels comprises nanoparticles.

In some embodiments, the method may further include the steps of: partitioning the first fluid into one or more portions; and contacting a portion of the one or more portions with one or more magnetic beads functionalized with an immobilized binding partner.

In some embodiments, the one or more properties of the free binding complex is one or more of a concentration of analyte, on-rate, off-rate, or binding affinity.

In some embodiments, any one of the first fluid, second fluid, and/or one or more portions are fluid droplets.

In some embodiments, the fluid droplets are manipulated using digital microfluidics (DMF) mediated droplet operations.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates a block diagram of an example of a digital microfluidic (DMF) system for performing solution-phase kinetic analysis in a DMF device (or cartridge), in accordance with an embodiment of the disclosure;

FIG. 2A and FIG. 2B illustrate schematic diagrams of an example of an LSPR sensor for analysis of analytes in the DMF device of the DMF system;

FIG. 3 illustrates a side view of an example of an LSPR sensor for analysis of analytes in the DMF device of the DMF system;

FIG. 4A and FIG. 4B illustrate cross-sectional views of a portion of a DMF device and including an example of an LSPR sensor on an optical fiber;

FIG. 5 illustrates a flow diagram of an example of a method of solution-phase kinetic analysis on a DMF device, in accordance with an embodiment of the disclosure;

FIG. 6A through FIG. 6F illustrate cross-sectional views of a portion of a DMF device and showing pictorially certain steps of the method shown in FIG. 5;

FIG. 7A and FIG. 7B illustrate an example of a sensorgram and a calibration curve, respectively, generated in the method shown in FIG. 5;

FIG. 8 illustrates a flow diagram of another example of a method of solution-phase kinetic analysis on a DMF device, in accordance with an embodiment of the disclosure;

FIG. 9A through FIG. 9J illustrate cross-sectional views of a portion of a DMF device and showing pictorially certain steps of the method shown in FIG. 8;

FIG. 10 illustrates a flow diagram of yet another example of a method of solution-phase kinetic analysis on a DMF device, in accordance with an embodiment of the disclosure; and

FIG. 11A through FIG. 11E illustrate cross-sectional views of a portion of a DMF device and showing pictorially certain steps of the method shown in FIG. 10.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

In some embodiments, the subject matter provides methods of solution-phase kinetic analysis on a digital microfluidic (DMF) device.

In some embodiments, the methods of solution-phase kinetic analysis on a DMF device provide a DMF system including a DMF device (or cartridge), a controller, a DMF interface, a detection system, one or more magnets, and one or more thermal control mechanisms and wherein the DMF device (or cartridge) may further include one or more electrode arrangements.

In some embodiments, the methods of solution-phase kinetic analysis on a DMF device provide a DMF device (or cartridge) including one or more electrode arrangements and wherein each of the electrode arrangements may include, but is not limited to, any arrangements (e.g., lines, paths, arrays) of droplet operations electrodes (i.e., electrowetting electrodes), one or more reservoirs, and one or more localized surface plasmon resonance (LSPR) sensors.

In some embodiments, the methods of solution-phase kinetic analysis on a DMF device provide a first method describing performing a calibration of signal vs. concentration, then assessing the concentration of the free analyte A in the sample by comparing it to a calibration curve (i.e., quantitation+screening solution samples).

In some embodiments, the methods of solution-phase kinetic analysis on a DMF device provide a second method using magnetically responsive beads having an binding partner B on their surface to capture any free analyte A and wherein the magnetically responsive beads can be immobilized as needed.

In some embodiments, the methods of solution-phase kinetic analysis on a DMF device provide a third method describing a magnetic bead-free process that uses fluorescently tagged antibodies and confocal microscopy (i.e., confocal microscope and probes).

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” refers to an amount that is near the stated amount by 10%, 5%, or 1%, including increments therein.

As used herein, the term “about” in reference to a percentage refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

### DMF System for Performing Solution-Phase Kinetic Analysis

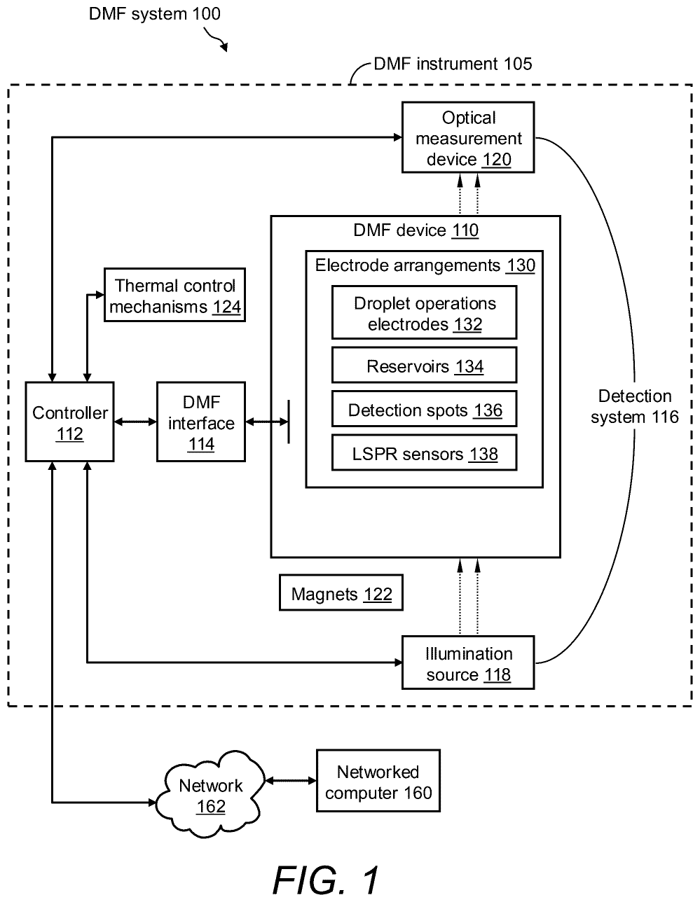

Referring now to FIG. 1 is a block diagram of an example of a DMF system 100 for performing solution-phase kinetic analysis in a DMF device (or cartridge), in accordance with an embodiment of the disclosure. In this example, DMF system 100 may include a DMF instrument 105. Further, DMF instrument 105 may house a DMF device (or cartridge) 110 along with any supporting components. DMF device 110 of DMF system 100 may be, for example, any fluidics device or cartridge, microfluidic device or cartridge, digital microfluidic (DMF) device or cartridge, droplet actuator, flow cell device or cartridge, and the like. In various embodiments, DMF system 100 provides DMF device 110 that may support automated processes to manipulate, process, and/or analyze biological materials.

DMF device 110 may be provided, for example, as a disposable and/or reusable device or cartridge. DMF device 110 may be used for processing biological materials. Generally, DMF device 110 may facilitate DMF capabilities for fluidic actuation including droplet transporting, merging, mixing, splitting, dispensing, diluting, agitating, deforming (shaping), and other types of droplet operations. Applications of these DMF capabilities may include, for example, sample preparation and waste removal. In one example, the DMF capabilities of DMF device 110 of DMF system 100 may be used to perform assays, such as, but not limited to, PCR protocols. For example, the DMF capabilities of DMF device 110 and DMF system 100 may be used for processing a patient sample and performing an assay. In DMF system 100, DMF device 110 may be provided, for example, as a disposable and/or reusable cartridge.

DMF system 100 may further include a controller 112, a DMF interface 114, a detection system 116, one or more magnets 122, and one or more thermal control mechanisms 124. Controller 112 may be electrically coupled to the various hardware components of DMF system 100, such as to DMF device 110, detection system 116, magnets 122, and thermal control mechanisms 124. In particular, controller 112 may be electrically coupled to DMF device 110 via DMF interface 114, wherein DMF interface 114 may be, for example, a pluggable interface for connecting mechanically and electrically to DMF device 110.

Detection system 116 may be any detection mechanism that can be used to accurately determine the presence or absence of a defined analyte and/or target component in different materials and to sensitively quantify the amount of analyte and/or target components present in a sample. Detection system 116 may be, for example, an optical measurement system that includes an illumination source 118 and an optical measurement device 120. For example, detection system 116 may be a fluorimeter that provides both excitation and detection.

The illumination source 118 may be, for example, a light source for the visible range (400-800 nm), such as, but not limited to, a white light-emitting diode (LED), a halogen bulb, an arc lamp, an incandescent lamp, lasers, and the like. Illumination source 118 is not limited to a white light source. Illumination source 118 may be any color light that is useful in DMF system 100. Optical measurement device 120 may be used to obtain light intensity readings. Optical measurement device 120 may be, for example, a charge coupled device, a photodetector, a spectrometer, a photodiode array, or any combinations thereof. Further, DMF system 100 is not limited to one detection system 116 only (e.g., one illumination source 118 and one optical measurement device 120 only). DMF system 100 may include multiple detection systems 116 (e.g., multiple illumination sources 118 and/or multiple optical measurement devices 120) to support multiple detection spots 136.

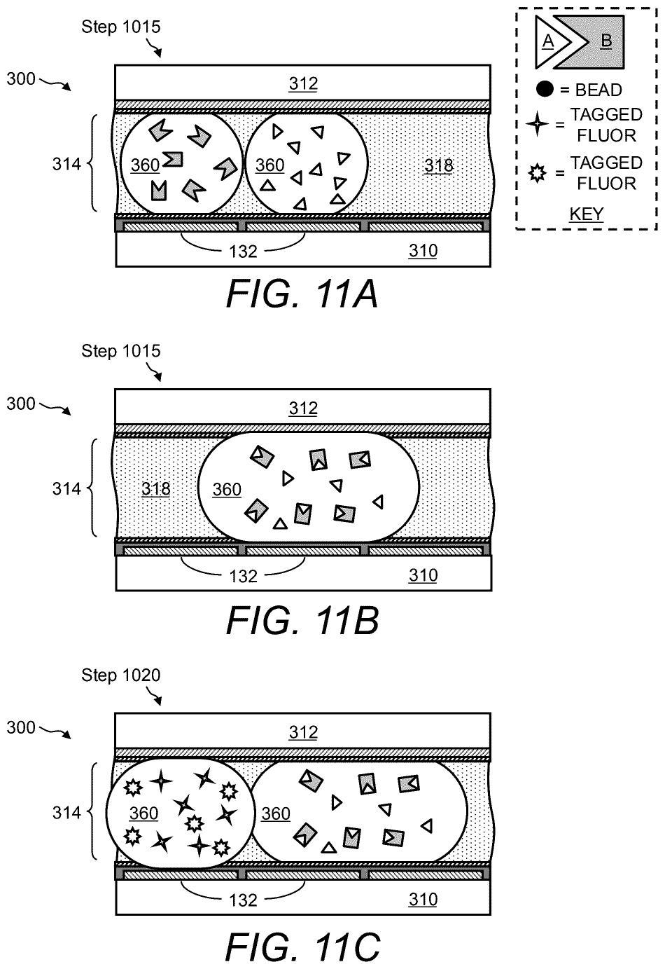

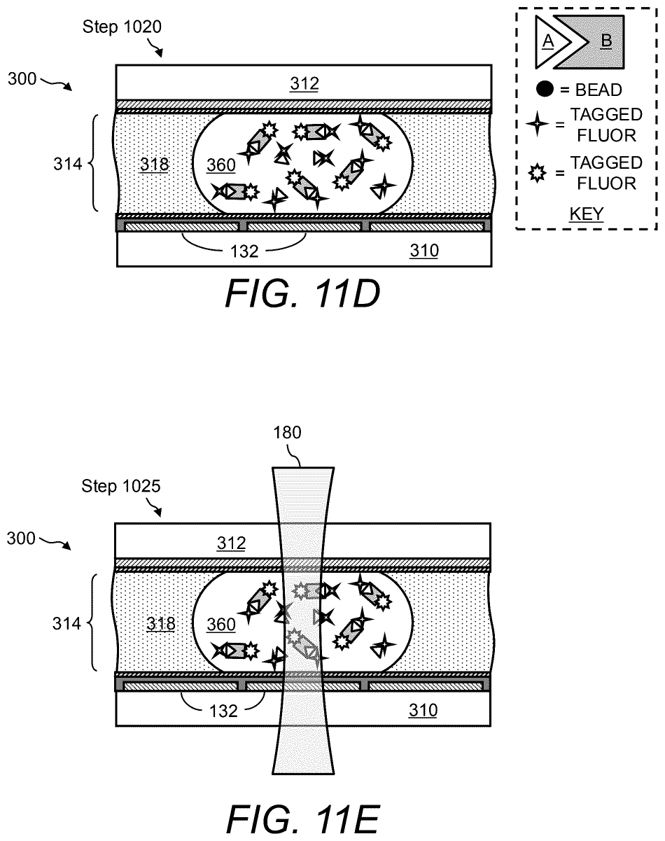

In another example, detection system 116 may include confocal microscopy (see FIG. 11E). Confocal microscopy is a specialized form of standard fluorescence microscopy (also called widefield fluorescence microscopy) that uses particular optical components to generate high-resolution images of material stained with fluorescent probes.

Controller 112 may, for example, be a general-purpose computer, special purpose computer, personal computer, microprocessor, or other programmable data processing apparatus. Controller 112 may provide processing capabilities, such as storing, interpreting, and/or executing software instructions, as well as controlling the overall operations of DMF system 100. The software instructions may comprise machine readable code stored in non-transitory memory that is accessible by the controller 112 for the execution of the instructions. Controller 112 may be configured and programmed to control data and/or power aspects of DMF system 100. Further, data storage (not shown) may be built into or provided separate from controller 112.

Thermal control mechanisms 124 may be any mechanisms for controlling the operating temperature of DMF cartridge 110. Examples of thermal control mechanisms 124 may include Peltier elements and resistive heaters.

Generally, controller 112 may be used to manage any functions of DMF system 100. For example, controller 112 may be used to manage the operations of, detection system 116 (e.g., illumination source 118 and optical measurement device 120), magnets 122, thermal control mechanisms 124, and any other instrumentation (not shown) in relation to DMF device 110. Magnets 122 may be, for example, permanent magnets and/or electromagnets. In the case of electromagnets, controller 112 may be used to control the electromagnets 122. Further, with respect to DMF device 110, controller 112 may control droplet manipulation by activating/deactivating electrodes.

In other configurations of DMF system 100, the functions of controller 112, detection system 116 (e.g., illumination source 118 and optical measurement device 120), magnets 122, thermal control mechanisms 124, and/or any other instrumentation may be integrated directly into DMF device 110 rather than provided separately from DMF device 110.

Optionally, DMF instrument 105 may be connected to a network. For example, controller 112 may be in communication with a networked computer 160 via a network 162. Networked computer 160 may be, for example, any centralized server or cloud-based server. Network 162 may be, for example, a local area network (LAN), a wide area network (WAN), or a cellular network for connecting to the internet.

Further, DMF device 110 of DMF system 100 may include one or more electrode arrangements 130. Each of the electrode arrangements 130 may include, but is not limited to, any arrangements (e.g., lines, paths, arrays) of droplet operations electrodes 132 (i.e., electrowetting electrodes). Droplet operations electrodes 132 may be used to fluidly connect any arrangements of one or more reservoirs 134. Further, certain droplet operations electrodes 132 may be designated as detection spots 136. In one example, illumination source 118 and optical measurement device 120 may be arranged with respect to detection spots 136 of DMF device 110.

Reservoirs 134 may be any fluid sources integrated with or otherwise fluidly coupled to DMF device 110. Reservoirs 134 may include any number and/or arrangements of, for example, on-cartridge reservoirs, off-cartridge reservoirs, blister packs, fluid ports, and the like, and any combinations thereof. Reservoirs 134 may be used to manage any liquids, such as reagents, buffers, sample volumes, and the like, needed to support any processes of DMF device 110. On-cartridge reservoirs 134, for example, may be formed of particular arrangements of droplet operations electrodes 132.

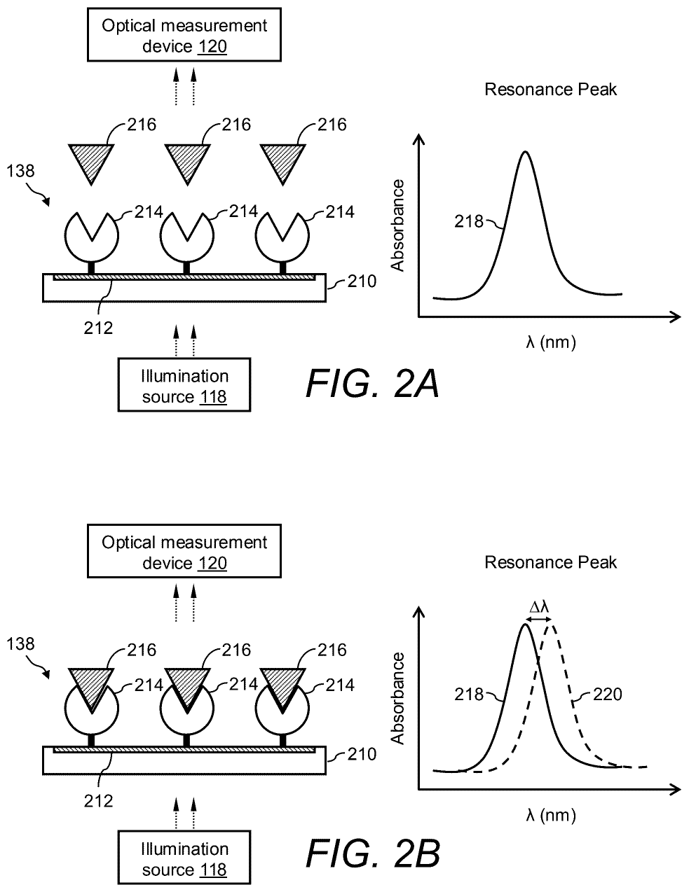

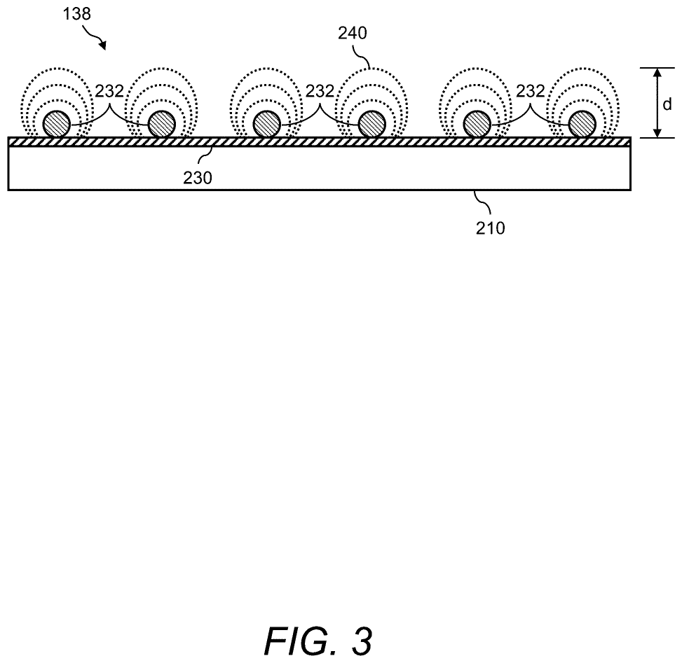

Further, each of the electrode arrangements 130 may include one or more localized surface plasmon resonance (LSPR) sensors 138. LSPR sensors are also referred to as surface plasmon resonance (SPR) sensors. Generally, LSPR sensors (e.g., LSPR sensors 138) may be functionalized for (1) detecting, for example, certain molecules (e.g., target analytes) and/or chemicals in the sample, and (2) analysis of analytes; namely, for measuring binding events in real time to extract ON-rate information, OFF-rate information, and/or affinity information. More details of examples of LSPR sensors are shown and described below with reference to FIG. 2A, FIG. 2B, and FIG. 3.

Referring now to FIG. 2A and FIG. 2B are schematic diagrams of an example of an LSPR sensor 138 for analysis of analytes in DMF device 110 of DMF system 100. Generally, LSPR is label-free interaction analysis in real-time. However, LSPR can also be used with labels to enhance the signal. The basic structure of the assay is a sensor chip (e.g., LSPR sensor 138) that includes a glass or plastic substrate with a surface that produces LSPR, such as a collection of discrete nanostructures distributed on a surface, or a continuous film that has nano-sized features formed therein, as shown, for example, in FIG. 3. Then, one of two binding partners is immobilized on the surface of the sensor. In LSPR, the “ligand” is the binding partner that is immobilized on the surface of the sensor. The “analyte” is what flows in solution over the ligand on the surface of the sensor. When the analyte binds to the ligand, it changes the optical properties of the surface of the sensor, which is measurable in real time.

In one example, LSPR sensor 138 may include a substantially transparent or opaque substrate 210, such as a glass, plastic, or TPE substrate. Namely, substrate 210 may be substantially transparent when LSPR sensor 138 is used in a transmission mode configuration. In another example, substrate 210 may be opaque when LSPR sensor 138 is used in a reflection mode configuration. An LSPR sensor layer 212 is provided atop substrate 210. LSPR sensor layer 212 can be, for example, a gold film that includes certain nanostructures (see FIG. 3) that create an LSPR effect or may be a plurality of plasmonic nanoparticles. LSPR sensor layer 212 is functionalized with one or more capture molecules 214. In one example, capture molecules 214 are ligands that are immobilized on the surface of LSPR sensor layer 212. In this example, the ligand is one of two binding partners, the other binding partner being a target analyte 216, wherein the target analyte 216 flows in solution over the capture molecules 214 as shown in FIG. 2A. By contrast, FIG. 2B shows the target analytes 216 binding to capture molecules 214. This binding may be referred to as a binding event.

Referring now again to FIG. 2A, a plot 218 is provided that indicates the optical absorbance peak of LSPR sensor layer 212 prior to a binding event occurring. That is, plot 218 shows the peak position or intensity prior to target analytes 216 binding to capture molecules 214 in LSPR sensor 138. Referring now to FIG. 2B, the change in peak position or intensity that is induced by binding of the target analytes 216 to the capture molecules 214 can be monitored in real time. For example, by comparing the peak position prior to binding (i.e., plot 218) with the peak position after binding (i.e., a plot 220). Generally, in LSPR sensor 138, as analytes bind to the surface, the resonance peak of the light will shift to a higher wavelength, which is measurable in real time.

Referring now to FIG. 3 is an example of a LSPR sensor 138 wherein the LSPR sensor layer includes nanostructures that can produce LSPR. For example, FIG. 3 shows a side view of an LSPR sensor 138 that includes colloidal-shaped nanostructures. Namely, LSPR sensor 138 includes the substantially transparent substrate 210, such as a glass, plastic, or TPE substrate. Next, an adhesive layer 230 is provided atop substrate 210. Next, an array or arrangement of nanoparticles 232 are provided on the surface of adhesive layer 230. In one example, nanoparticles 232 can be metal nanoparticles (e.g., gold, silver, copper nanoparticles, or combinations thereof) that are immobilized on or linked to substrate 210 using physical or chemical coupling, such as using adhesive layer 230. Nanoparticles 232 can be, for example, from about 1 nm to about 1000 nm in various dimensions and in various shapes, such a spheres, stars, rice, cubes, cages, urchins, rods, and the like. Next, capture molecules 214 (not shown) as described in FIG. 2A and FIG. 2B can be immobilized on nanoparticles 232. In this example, the LSPR sensor 138 emits an optical signal 240 that is present very close to the surface. In one example, optical signal 240 can be detected within a distance d of from about 0 nm to about 100 nm from the surface.

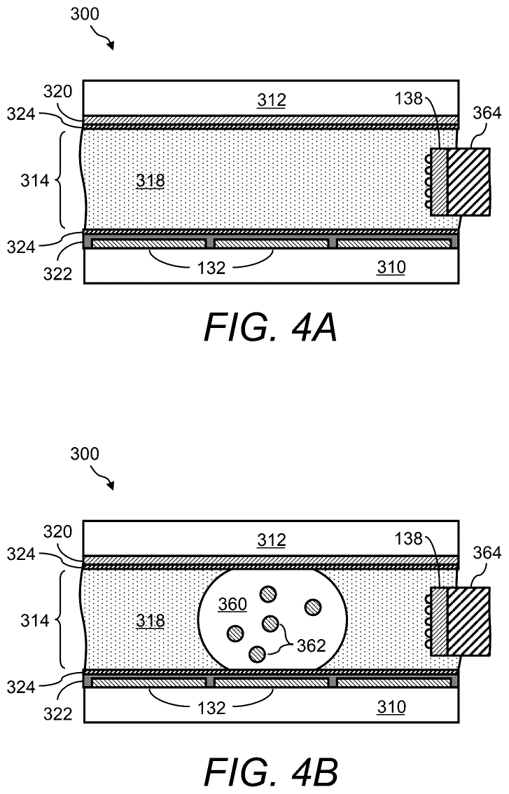

Referring now to FIG. 4A and FIG. 4B is cross-sectional views of a DMF structure 300. The formation of DMF device 110 of DMF system 100 may be based generally on DMF structure 300. Further, FIG. 4A and FIG. 4B show DMF structure 300 including an example of an LSPR sensor 138 on an optical fiber 364.

DMF structure 300 may be used to form any arrangements (e.g., lines, paths, arrays) of droplet operations electrodes 132 (i.e., electrowetting electrodes). DMF structure 300 may include a bottom substrate 310 and a top substrate 312 separated by a droplet operations gap 314. Droplet operations gap 314 may contain filler fluid 318, such as silicone oil or hexadecane.

Bottom substrate 310 may be, for example, a silicon substrate or a printed circuit board (PCB). Bottom substrate 310 may include an arrangement of droplet operations electrodes 132 (e.g., electrowetting electrodes). Droplet operations electrodes 132 may be formed, for example, of copper, gold, or aluminum. A dielectric layer 322 (e.g., parylene coating, silicon nitride) may be atop droplet operations electrodes 132.

Top substrate 312 may be, for example, a glass or plastic substrate. Top substrate 312 may include a ground reference electrode 320. In one example, ground reference electrode 320 may be formed of indium tin oxide (ITO) and wherein ITO is substantially transparent to light. Further, a hydrophobic layer 324 may be provided on both the side of bottom substrate 310 and the side of top substrate 312 that is facing droplet operations gap 314. Examples of hydrophobic materials or coatings may include, but are not limited to, polytetrafluoroethylene (PTFE), Cytop, Teflon™ AF (amorphous fluoropolymer) resins, FluoroPel™ coatings, silane, and the like.

Droplet operations may be conducted atop droplet operations electrodes 132 on a droplet operations surface. For example, droplet operations may be conducted atop droplet operations electrodes 132. That is, an aqueous droplet 360 may be present in droplet operations gap 314 of DMF structure 300, as shown, for example, in FIG. 4B. In one example, droplet 360 may be a droplet of a blood sample (or fraction thereof) or a saliva sample to be evaluated. In another example, droplet 360 may be a reagent droplet for conducting an assay. In yet another example, droplet 360 may be a reaction droplet that may or may not include a target analyte of interest (e.g., analyte 362). Filler fluid 318 may fill droplet operations gap 314 and surround droplet 360.

In this example, optical fiber 364 with the LSPR sensor 138 is arranged in the droplet operations gap 314 of DMF structure 300. Accordingly, aqueous droplet 360 may come into contact with LSPR sensor 138 using droplet operations.

### Process/System Configurations for Solution-Phase Kinetic Analysis on a DMF Device

Using, for example, DMF device 110 of DMF system 100 shown in FIG. 1, a first process and/or system configuration for performing solution-phase kinetic analysis, may be as follows. Further, references below to “A” and “B” means analyte A (or binding partner A) and analyte B (or binding partner B). Analytes A and B (i.e., binding partners A and B) may consist of biomolecules such as antibodies, antibody fragments, recombinant proteins, oligonucleotides, lipids, small molecules, viruses and virus like particles (VLP) or whole cells. By performing solution-phase kinetic analysis on DMF enables analysis of biomolecules in crude media, such as lysate, serum, plasma, hybridoma supernatant and B-cell media can be analyzed.

In some embodiments, analyte A may be an antibody and analyte B may be its antigen. For example, analyte A may be an anti-SARS-COV-2 Spike RBD antibody and analyte B may be the SARS-COV-2 Spike RBD recombinant protein. In some embodiments, analyte A may be a virus like particle (VLP) and analyte B may be an antibody specific to one of the protein subunits on the VLP. In some embodiments, analyte A may be a small molecule and analyte B may be a recombinant protein.

Generally, by performing solution-phase kinetic analysis with these biomolecules strong affinity values (KDs), in the low pM to fM range, may be detected, which may be challenging for surface-based kinetic analysis.

An example of the process and/or system configuration includes using, for example, DMF device 110, the readout for the solution-phase may be an SPR sensor, such as LSPR sensor 138 installed in DMF structure 300 as shown in FIG. 4A and FIG. 4B. In this example, a calibration of signal vs. concentration is performed, then the concentration of the free analyte in the sample is assessed by comparing it to the curve (i.e., quantitation+screening solution samples). Compared with conventional methods of performing affinity measurements, providing a DMF-based test may be used to reduce sample volume. Specifically, a DMF-based device can manipulate fluid volumes in the picoliter to nanoliter range which may be challenging for flow-based systems. By reducing the sample volume required for a given analysis or measurement, DMF enables lower costs.

Another example of the process and/or system configuration includes using, for example, DMF device 110, the process can aliquot a sample and measure it at several time points per concentration. This allows measurement of the ON-rate (K_{ON}) and calculation of the OFF-rate (K_{OFF}) in addition to the affinity value (K_{D})). A DMF-based device enables process automation and may consume very little sample volume per measurement such that more measurements can be made from a given sample vs. conventional instrumentation.

In yet another example the process and/or system configuration includes using, for example, DMF device 110, to create a quantitation standard curve, this is followed by a solution-phase experiment. From this standard curve, comprising binding and dissociation cycles, the ON-rate, OFF-rate and binding affinity can be directly measured. However, this is for a system in which one of analyte A or analyte B is attached to the surface of the SPR or LSPR sensor 138. Therefore, using this standard curve, kinetics may be obtained for the surface immobilized complexation, and the kinetics in solution are expected to be similar (i.e., the kinetics for the surface immobilized complex may be used to predict ideal experimental conditions for the solution kinetics measurement).

In yet another example the process and/or system configuration includes using, for example, DMF device 110, in which a secondary protein may be used as a tag to improve the limits of detection (LOD). In this example, after binding an analyte A to an analyte B immobilized on a surface of a SPR or LSPR sensor 138 (i.e., formation of an analyte A+analyte B complex on the sensor surface), a signal enhancer C (e.g., a secondary protein), may be introduced which bonds to the analyte A+analyte B complex. The signal enhancer C may be larger than analyte A such that the SPR shift introduced by binding of signal enhancer C is greater than the SPR shift introduced by the binding of analyte A to analyte B, while still being proportional to the amount of analyte A bound to analyte B. Additionally, a complementary probe (i.e., signal enhancer) having a metal nanoparticle attached may be used. This nanoparticle's localized surface plasmon resonance field will interfere with the field of the SPR sensor and enhance the signal shift induced by the binding. The nanoparticles may be solid gold, solid silver, composite, core-shell. Core-shell nanoparticles may have a metal core and metal shell or non-metal cores (e.g., silicon core) and metal shells. The nanoparticles may be different shapes such as spheres, urchins, prisms, decahedral and other shapes. Generally, the nanoparticles may be from about 10 nm to about 200 nm, or any size therebetween.

In yet another example, the process and/or system configuration includes using, for example, DMF device 110, in which the experimental protocol may be adjusted in real time to optimize and account for non-ideal behavior.

In an example, real time adjustment includes using the calibration curve data in the third example process and/or system configuration above to calculate the ON-rate, and using the ON-rate to determine the ideal sampling times required for the solution phase samples to get good results from the solution phase analysis.

Another example of real time adjustment includes using the calibration curve data in the third example process and/or system configuration above to determine whether to use a signal enhancer, such as, for example, a secondary protein to amplify signal.

In yet another example of real time adjustment, if the solution phase sample measurements indicate that the reaction is far from equilibrium (i.e., the concentration of unbound analyte A is changing rapidly) then the system can automatically increase the sampling frequency. Conversely, if the reaction is approaching equilibrium, then the system can decrease the measurement (i.e., sampling) frequency and extend the experiment run time. This ensures sampling is performed at appropriate timepoints to produce a better kinetics estimation.

In yet another example of real time adjustment, the sample timing may be automated by simulating how the concentration of analyte A changes with time based on the surface kinetics measured as part of the calibration curve in the third example process and/or system configuration above.

Using, for example, DMF device 110 of DMF system 100 shown in FIG. 1, a second process and/or system configuration for performing solution-phase kinetic analysis, may be as follows.

In an example, similar to the first process and/or system configuration, however, the assay utilizes a fluorescence based measurement and magnetic beads. This configuration benefits from low sample volume, automated time sampling, the ability to multiplex on-cartridge automatically, and the ability to automatically determine equilibrium is reached. Further, this configuration benefits from the ability to increase throughput with more sampling points. Moreover, this configuration benefits from improved LOD.

This bead-based measurement could utilize other forms of probes, such as radioactive probes, enzymes to create new reports, such as chemiluminescence or oxidizing species. The oxidizing species could then be used to induce a color change, or etch nanoparticles to induce a color change, etc.

Using, for example, DMF device 110 of DMF system 100 shown in FIG. 1, a third process and/or system configuration for performing solution-phase kinetic analysis, may be as follows.

In an example, similar to the second process and/or system configuration, however, the system may include both DMF and confocal fluorescence microscopy. In this configuration, a dilution series of analyte B is mixed with a defined concentration of analyte A. Fluorescently tagged anti-analyte A and anti-analyte B are introduced, each emitting light at different wavelengths. The fluorescent signal is measured and autocorrelated signals for each fluorophore are generated along with cross-correlated signals. Fluorescence correlation spectroscopy (FCS) may be used to assess the size of the molecule bound to each anti-B and anti-A fluorophore. This may be used to separate the signal of the analyte A+analyte B complex from the free analyte A (or free fluorophores). In the case where analyte A and analyte B are free in solution, there will be high autocorrelations of analyte A and analyte B but a low cross correlation. As analyte A and analyte B bind (i.e., formation of an analyte A+analyte B complex), the cross correlation will increase. Using a model, these correlation factors can be used to quantify unbound (or free) analyte A, unbound (or free) analyte B, and analyte A+analyte B complex concentrations. The advantage of this third configuration vs. the second configuration above is that no magnetic beads are required. Additional versions of this third configuration may include the following: (1) use of only one fluorophore (enabling assessment of the anti-analyte A, anti-analyte A+analyte A complex, anti-analyte A+analyte A+analyte B complex) to simplify the optical requirements of the system; and (2) introducing the fluorophores during mixing of analyte A and analyte B to measure real-time binding.

### Methods of Solution-Phase Kinetic Analysis on a DMF Device

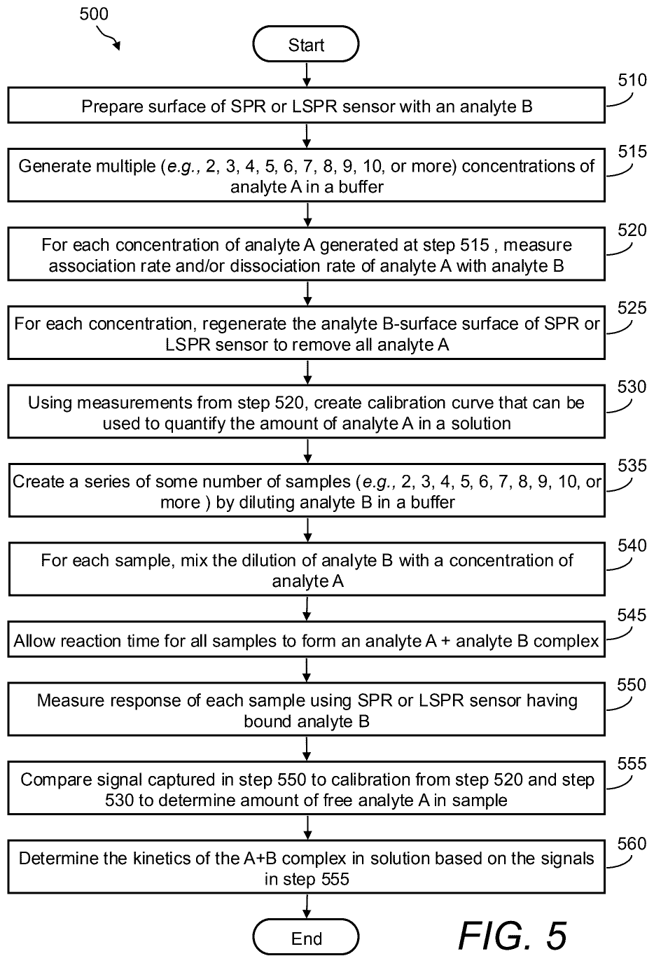

Referring now to FIG. 5 is a flow diagram of method 500, which is an example of a method of solution-phase kinetic analysis on a DMF device, such as DMF device 110 of DMF system 100 shown in FIG. 1, in accordance with an embodiment of the disclosure. In preparation for executing method 500, a certain DMF device 110 may be selected and then installed in DMF instrument 105. Then, any SPR or LSPR sensors 138 therein (see FIG. 4A and FIG. 4B) may be cleaned using a cleaning agent (e.g., hydrogen chloride (HCl)). Method 500 may include, but is not limited to, the following steps.

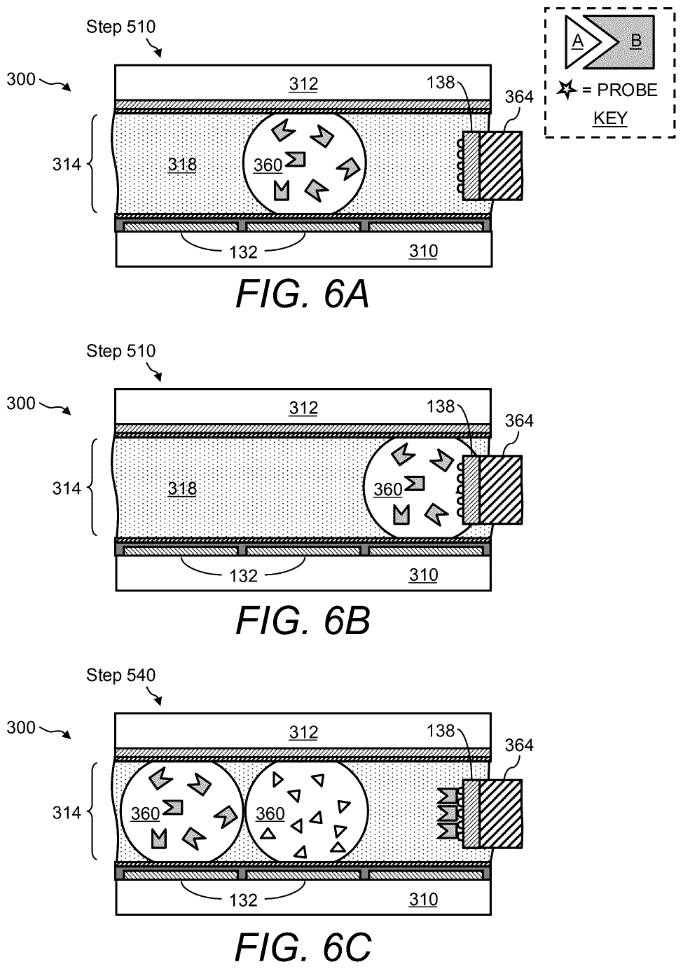

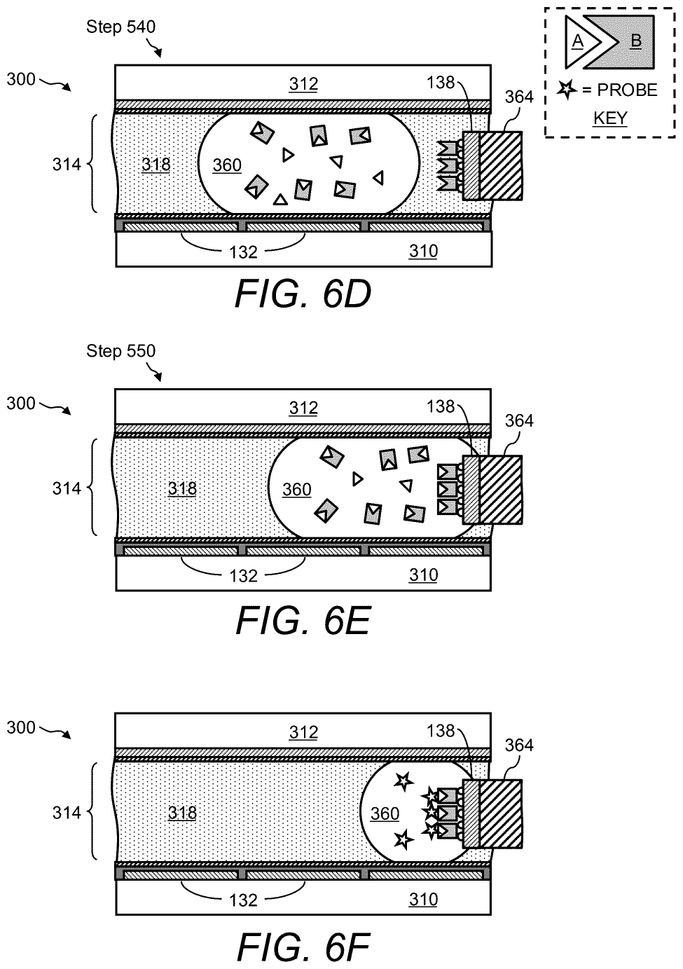

At step 510, the surface of the SPR or LSPR sensor 138 is prepared with an analyte B. This is done by immobilizing analyte B on SPR or LSPR sensor 138, as shown, for example, in FIG. 6A and FIG. 6B. Immobilizing analyte B on the surface may be accomplished through many methods, such as, for example, via chemisorption of thiols on a metal surface. In another example, an intermediate linker disposed on the surface of the SPR or LSPR sensor 138 may be used. The intermediate linker may include a reactive functional group such as, for example, a carboxyl or carboxylic acid that may be configured to react with analyte B.

At step 515, multiple (e.g., 2, 3, 4, 5, 6, 7, 8, 9, 10, or more) concentrations of analyte A in a buffer are generated. These concentrations may be done external to the cartridge and introduced or may be automatically produced in the droplet operations gap by using DMF procedures. Each concentration of analyte A is then contacted with the SPR or LSPR sensor 138 comprising analyte B. Analyte B may be immobilized to surface of the SPR or LSPR sensor 138.

At step 520, for each concentration of analyte A generated at step 515, the association rate and/or dissociation rate of analyte A with analyte B is measured. In this step, each of the calibration steps may be done with the secondary tag.

At step 525, after each measurement, analyte B on the surface of the SPR or LSPR sensor 138 is regenerated using a buffer to remove analyte A.

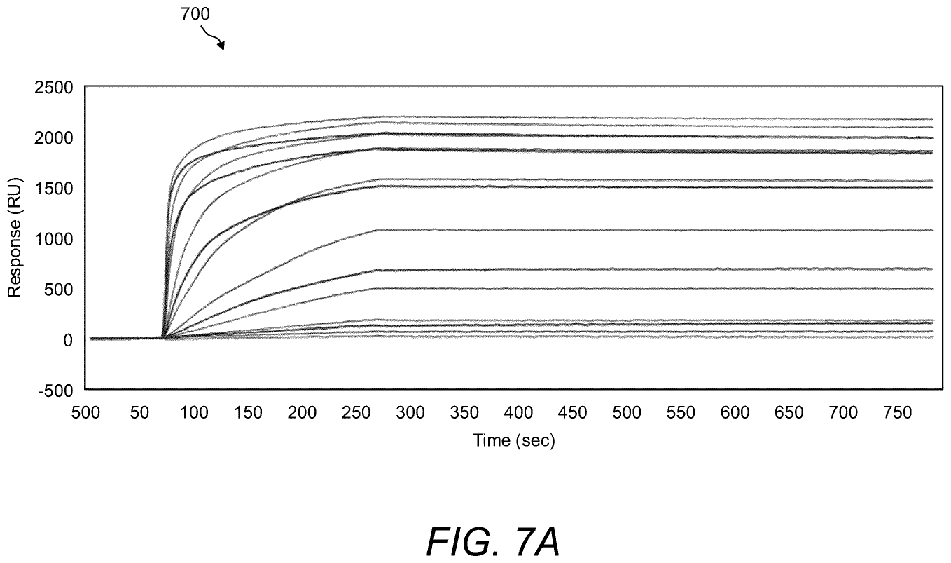

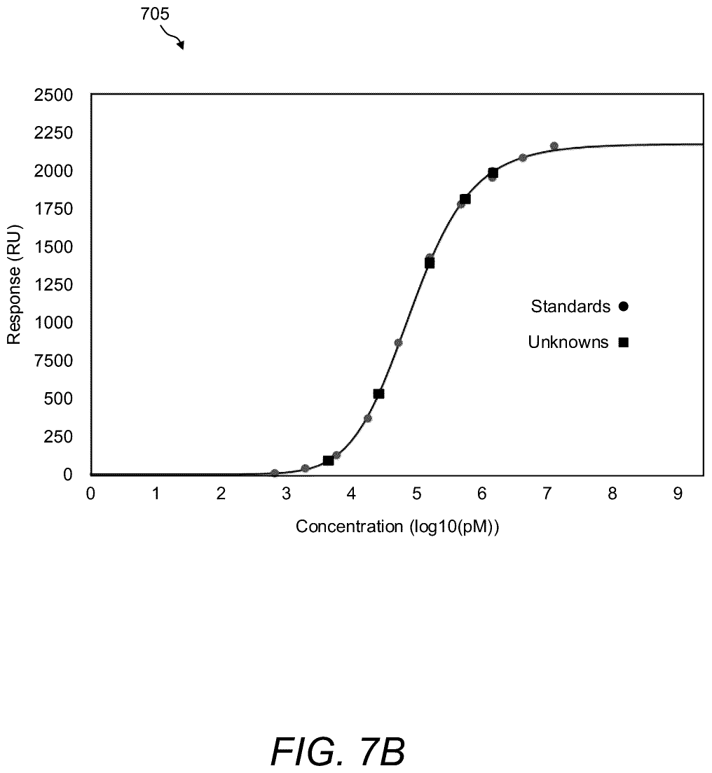

At step 530, using the measurements from step 520, a calibration curve is generated that may be used to quantify the amount of analyte A in a solution. The calibration curve may also allow for determination of the association and/or dissociation reaction constants for the reaction of analyte A with analyte B in solution. For example, FIG. 7A and FIG. 7B illustrate an example of a sensorgram 700 and a calibration curve 705, respectively, generated here in step 530. Sensorgram 700 shows an example of measured signals (i.e., sensorgram). Further, in the event that each of the calibration steps in 520 are done with a secondary tag, the output of this step 530 has the tag.

At step 535, a series of some number of samples (e.g., 2, 3, 4, 5, 6, 7, 8, 9, 10, or more) is created by diluting analyte B in a buffer. For example, this may be a serial dilution having a 1:1, 1:2, 1:3, 1:4, 1:5, 1:6, 1:7, 1:8, 1:9, 1:10, or other ratio.

At step 540, for each sample, the dilution of analyte B is mixed with a concentration of analyte A. The concentration of analyte A is constant for all analyte samples while the concentration of analyte B is variable. This step is shown, for example, in FIG. 6C and FIG. 6D.

At step 545, a certain amount of reaction time is allowed for all samples to form an analyte A+analyte B complex (i.e., equilibration). In one example, controller 112 may be used to automatically determine the amount of reaction time needed based on the values obtained in steps 520 and 530 which is typically determined from the surface kinetic constants (ON-rate, OFF-rate, and equilibrium (K_{D}) constants). Generally, the reaction time may be about 1/K_{D}). The reaction time may be, for example, minutes, hours, or tens of hours.

At step 550, using SPR or LSPR sensor 138 having bound analyte B, the response of each sample is measured, and then regenerate SPR or LSPR sensor 138 after each measurement using a buffer. This step is shown, for example, in FIG. 6E.

At step 555, the signal generated by the capture of analyte A on the surface of the SPR or LSPR sensor 138 from step 550 is compared to the calibration from steps 520 and 530 to determine the amount of free analyte A in the sample.

At step 560, the kinetics of the analyte A+analyte B complex in solution is determined based on the comparison performed in step 555.

Further, certain variations of method 500 are possible. In one example, in the event that a secondary tag is used, this would adjust both the calibration steps 520 and 530 as well as the analysis step 555. In another example, a signal enhancer C may be introduced at the surface of the SPR or LSPR sensor 138. Signal enhancer C may bind with the analyte A+analyte B complex to amplify the signal (i.e., induce a greater shift in the plasmon resonance signal), as shown, for example, in FIG. 6F. Signal enhancer C may be a larger molecule that selectively bonds to analyte A, or a nanoparticle.

Referring now to FIG. 6A through FIG. 6F are cross-sectional views of DMF structure 300 that show pictorially steps of method 500 shown in FIG. 5. For example, FIG. 6A and FIG. 6B show pictorially an example of step 510 of method 500. Next, FIG. 6C and FIG. 6D show pictorially an example of step 540 of method 500. Next, FIG. 6E shows pictorially an example of step 550 of method 500. Next, FIG. 6F shows pictorially an example of one variation of method 500.

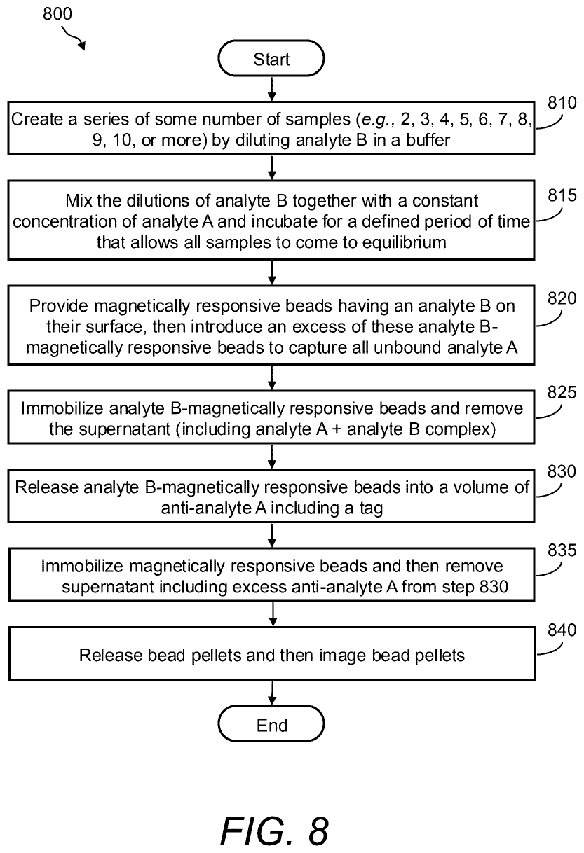

Referring now to FIG. 8 is a flow diagram of an example of a method 800, which is another example of a method of solution-phase kinetic analysis on a DMF device, such as DMF device 110 of DMF system 100 shown in FIG. 1, in accordance with an embodiment of the disclosure. Here, steps 510, 515, 520, 525, and 530 of method 500 shown in FIG. 5 may be performed before proceeding to method 800. Method 800 may include, but is not limited to, the following steps.

At step 810, a series of some number of samples (e.g., 2, 3, 4, 5, 6, 7, 8, 9, 10, or more) is created by diluting analyte B in a buffer. For example, this may be a serial dilution having a 1:2, 1:3, 1:4, 1:5, 1:6, 1:7, 1:8, 1:9, 1:10, or other ratio.

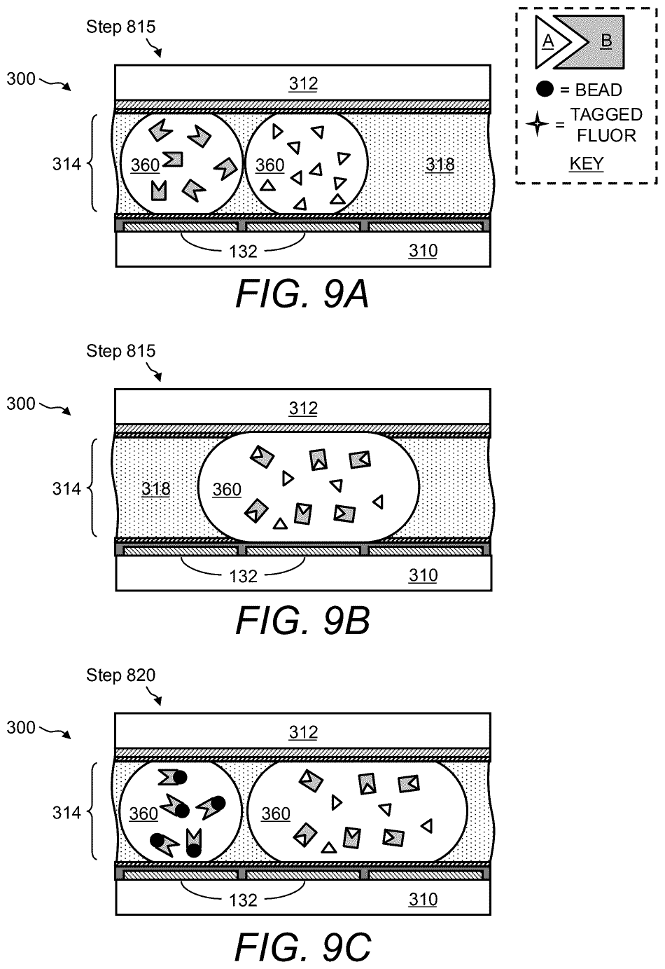

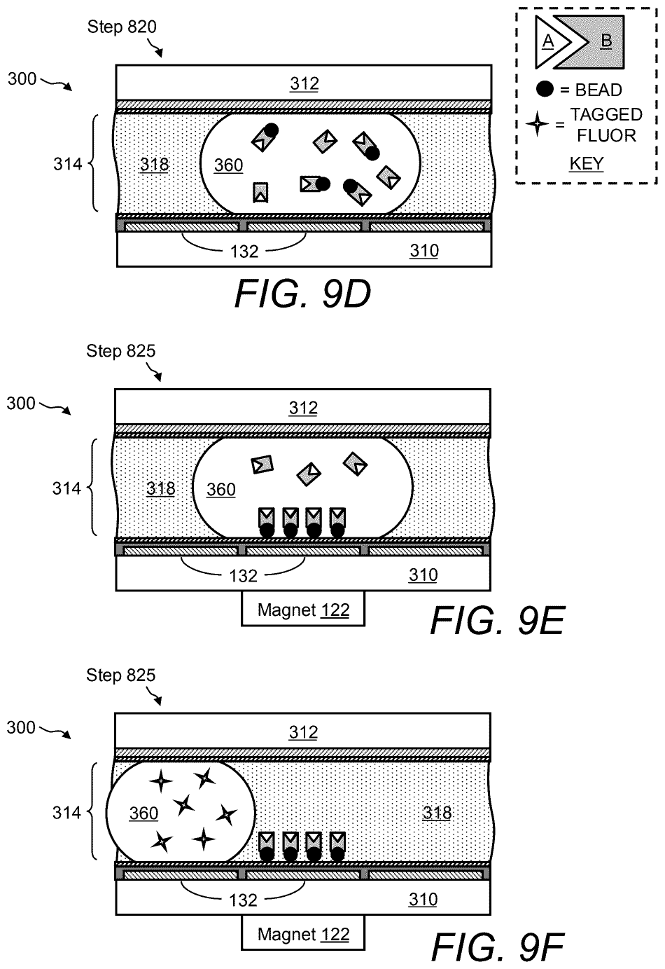

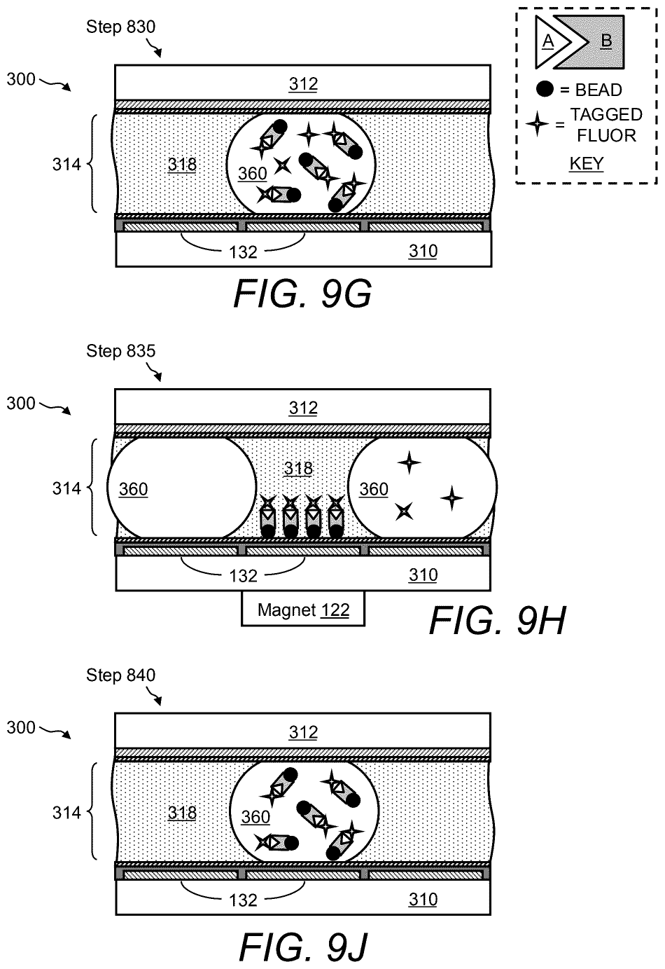

At step 815, the dilutions of analyte B are mixed together with a constant concentration of analyte A and then incubated for a defined period of time, as shown, for example, in FIG. 9A and FIG. 9B. Time is allowed for all samples to come to equilibrium (i.e., formation of an analyte A+analyte B complex). For example, the time to reach equilibrium may be minutes, hours, or tens of hours.

At step 820, magnetically responsive beads are provided that have analyte B disposed on their surface, then an excess of these analyte B-magnetically responsive beads are introduced to capture unbound analyte A, as shown, for example, in FIG. 9C and FIG. 9D.

At step 825, the analyte B-magnetically responsive beads are immobilized using a magnet 122 of DMF device 110 and then the supernatant (including the analyte A+analyte B complex) is removed, as shown, for example, in FIG. 9E and FIG. 9F. Immobilization may occur by positioning magnet 122 in close proximity to DMF structure 300.

At step 830, the analyte B-magnetically responsive beads are released into a volume of anti-analyte A comprising a tag, as shown, for example, in FIG. 9G. The concentration of anti-analyte A may be in excess when compared with the analyte B-magnetically responsive beads. This tag may be fluorescent, luminescent, or otherwise capable of producing an electromagnetic signal.

At step 835, the analyte B-magnetically responsive beads are immobilized using magnet 122 of DMF system 100 and then the supernatant (including excess anti-analyte A from step 830) is removed, as shown, for example, in FIG. 9H. Again, immobilization may occur by positioning magnet 122 in close proximity to DMF structure 300.

At step 840, the analyte B-magnetically responsive beads are released into a buffer solution and then imaged, as shown, for example, in FIG. 9J. The signal may be proportional to the amount of unbound analyte A from step 815.

Referring now to FIG. 9A through FIG. 9J is cross-sectional views of DMF structure 300 that show pictorially certain steps of method 800 shown in FIG. 8. For example, FIG. 9A and FIG. 9B show pictorially step 815 of method 800. Next, FIG. 9C and FIG. 9D show pictorially step 820 of method 800. Next, FIG. 9E and FIG. 9F show pictorially step 825 of method 800. Next, FIG. 9G shows pictorially step 830 of method 800. Next, FIG. 9H shows pictorially step 835 of method 800. Next, FIG. 9J shows pictorially step 840 of method 800.

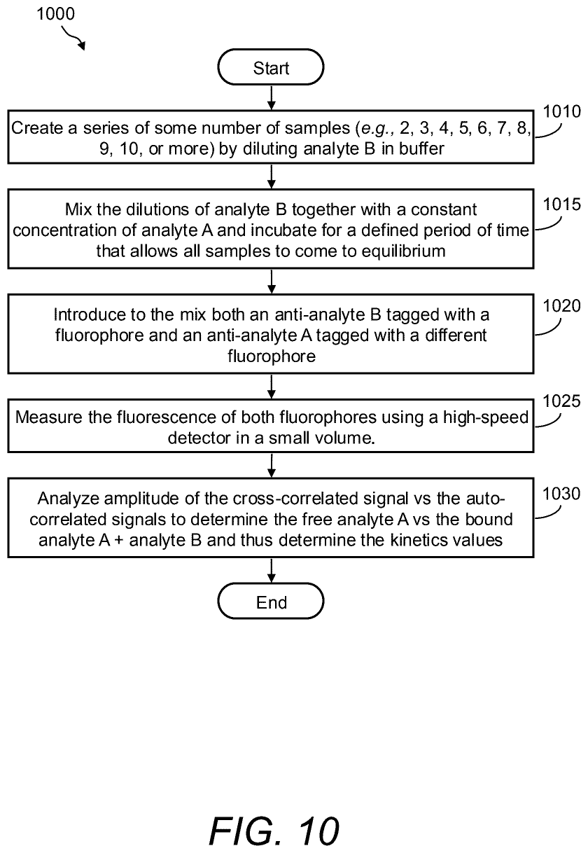

Referring now to FIG. 10 is a flow diagram of an example of a method 1000, which is yet another example of a method of solution-phase kinetic analysis on a DMF device, such as DMF device 110 of DMF system 100 shown in FIG. 1, in accordance with an embodiment of the disclosure. Here, steps 510, 515, 520, 525, and 530 of method 500 shown in FIG. 5 may be performed before proceeding to method 1000.

Further, method 1000 describes a magnetic bead-free process that uses fluorescently tagged antibodies and confocal microscopy (i.e., confocal microscope and probes). Method 1000 may include, but is not limited to, the following steps.

At step 1010, a series of some number of samples (e.g., 2, 3, 4, 5, 6, 7, 8, 9, 10, or more) is created by diluting analyte B in a buffer. For example, this may be a serial dilution having a 1:2, 1:3, 1:4, 1:5, 1:6, 1:7, 1:8, 1:9, 1:10, or other ratio.

At step 1015, the dilutions of analyte B are mixed together with a constant concentration of analyte A and then incubated for a defined period of time, as shown, for example, in FIG. 11A and FIG. 11B. Time is allowed for all samples to come to equilibrium (i.e., formation of an analyte A+analyte B complex). For example, the time to reach equilibrium may be minutes, hours, or tens of hours.

At step 1020, both an anti-analyte B tagged with a fluorophore and an anti-analyte A tagged with a different fluorophore are introduced to the mix, as shown, for example, in FIG. 11C and FIG. 11D. For example, the anti-analyte B fluorophore may emit at 500 nm and the anti-analyte A fluorophore may emit at 700 nm. Generally, the fluorophores may emit at any wavelength (e.g., from about 300 nm to about 800 nm) provided that the fluorophores may be spectrally separated. However, minimal spectral overlap may be desired.

At step 1025, the fluorescence of both fluorophores is measured using a high-speed detector in a small volume. Here, the objective is to measure the fluorescent species as they enter and exit the detection volume and then use temporal correlation to assess the parameters of interest. The detection volume should thus be minimal to ensure that the number of fluorophores in the detection volume is low. For example, a typical diameter of the detection volume is about 300 nm (i.e., about 100 fL), but may be a small or larger diameter. The detector must be optimized for low-light to ensure a good limit of detection. Further, the detector should be capable of imaging at speeds up to about 1000 frames per second (fps).

These signals are autocorrelated and cross-correlated. The ratio of the autocorrelated and cross-correlated signals can be used to determine the amount of analyte A+analyte B complexes formed vs free A or B. For example, the volume may be probed using a confocal microscope 180 by which the fluorescence of the two tags may be measured, as shown, for example, in FIG. 11E.

At step 1030, the amplitude of the cross-correlated signal vs the auto-correlated signal of A is analyzed to determine the free A vs the bound analyte A+analyte B, and thus determine the kinetics values.

Further, certain variations of method 1000 are possible. In one example, analyte A and analyte B may be fluorescently tag before starting method 1000. This removes step 1020. In another example, the fluorescent tags may be added before step 1015. This allows for real-time measurement of the binding. In another example, method 1000 may use magnetically responsive beads similar to that described above in method 800.

Referring now to FIG. 11A through FIG. 11E is cross-sectional views of DMF structure 300 that show pictorially certain steps of method 1000 shown in FIG. 10. For example, FIG. 11A and FIG. 11B show pictorially step 1015 of method 1000. Next, FIG. 11C and FIG. 11D show pictorially step 1020 of method 1000. Next, FIG. 11E shows pictorially step 1025 of method 1000.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

What is claimed is:

1. A method for determining solution phase properties of a binding complex, the method comprising:
providing a first fluid comprising a known concentration of an analyte;
contacting the first fluid with a sensor comprising a known concentration of an immobilized binding partner, wherein the immobilized binding partner is bound to a surface of the sensor and is configured to bind the analyte to form an immobilized binding complex;
measuring a signal generated by the sensor in response to formation of the immobilized binding complex;
generating a calibration curve using the signal generated by sensor;
providing a second fluid comprising a known concentration of the analyte and a known concentration of free binding partner, wherein the free binding partner is configured to bind the analyte to form a free binding complex;
contacting the second fluid with the sensor comprising the immobilized binding partner thereby forming a second immobilized binding complex; measuring a second signal generated by the sensor in response to formation of the second immobilized binding complex; and
comparing the second signal to the calibration curve to determine one or more properties of the free binding complex.

2. The method of claim 1, wherein the analyte, the immobilized binding partner, and the free binding partner are biomolecules.

3. The method of claim 2, wherein the biomolecules are selected from the group consisting of: antibodies, antibody fragments, recombinant proteins, oligonucleotides, lipids, small molecules, viruses and virus like particles (VLP), and whole cells.

4. The method of any one of claims 3, wherein immobilized binding partner and free binding partner are the same or substantially the same.

5. The method of any one of claims 4, wherein the immobilized binding complex and free binding complex are the same or substantially the same.

6. The method of any one of claims 5, further comprising,
partitioning the second fluid into one or more portions;
contacting a portion of the one or more portions with the sensor comprising the immobilized binding partner thereby forming an additional immobilized binding complex;
measuring one or more additional signals generated by the sensor in response to formation of the additional immobilized binding complex; and
comparing the one or more additional signals to the calibration curve to determine one or more properties of the free binding complex.

7. The method of any one of claims 6, wherein the one or more properties of the free binding complex is one or more of a concentration of analyte, on-rate, off-rate, or binding affinity.

8. The method of claim 7, further comprising, determining a sampling rate using the one or more properties of the free binding complex.

9. The method of claim 7, wherein any one of the first fluid, second fluid, and/or one or more portions are fluid droplets.

10. The method of claim 9, wherein the fluid droplets are manipulated using digital microfluidics (DMF) mediated droplet operations.

11. The method of any one of claims 10, wherein the sensor is a SPR or LSPR sensor.

12. The method of claim 11, wherein the SPR or LSPR sensor comprises a surface having a nanostructured portion.

13. The method of claim 12, wherein the first binding partner is immobilized to the nanostructured portion.

14. The method of any one of claims 13, wherein the sensor is disposed within a gap of a microfluidic cartridge.

15. The method of claim 1, further comprising,
providing one or more signal enhancers configured to bind to the immobilized binding complex thereby enhancing the signal generated by the sensor; and
providing one or more signal enhancers configured to bind to the second immobilized binding complex thereby enhancing the second signal generated by the sensor.

16. The method of claim 6, further comprising,
providing one or more signal enhancers configured to bind to the immobilized binding complex thereby enhancing the signal generated by the sensor; and
providing one or more signal enhancers configured to bind to the additional immobilized binding complexes thereby enhancing the one or more additional signals generated by the sensor.

17. A method for determining solution phase properties of a binding complex, the method comprising:
providing a first fluid comprising a known concentration of an analyte and a known concentration of a free binding partner, wherein the free binding partner is configured to bind the analyte to form a free binding complex;
contacting the first fluid with one or more magnetic beads functionalized with an immobilized binding partner, wherein the immobilized binding partner is configured to bind the analyte to form an immobilized binding complex;
isolating the one or more magnetic beads;
contacting the one or more magnetic beads with a second fluid comprising one or more types of labels, wherein the one or more types of labels are configured to bind the immobilized binding complex thereby forming a labeled binding complex; and
measuring a signal generated by the labeled binding complex to determine one or more properties of the free binding complex.

18. The method of claim 17, wherein the analyte, the immobilized binding partner, and the free binding partner are biomolecules.

19. The method of claim 18, wherein the biomolecules are selected from the group consisting of: antibodies, antibody fragments, recombinant proteins, oligonucleotides, lipids, small molecules, viruses and virus like particles (VLP), and whole cells.

20. The method of any one of claims 19, wherein the one or more types of labels comprises fluorescent labels.

21. The method of any one of claims 19, wherein the one or more types of labels comprises nanoparticles.

22. The method of any one of claims 19, further comprising,
partitioning the first fluid into one or more portions; and
contacting a portion of the one or more portions with one or more magnetic beads functionalized with an immobilized binding partner.

23. The method of claim 22, wherein the one or more properties of the free binding complex is one or more of a concentration of analyte, on-rate, off-rate, or binding affinity.

24. The method of claim 23, wherein any one of the first fluid, second fluid, and/or one or more portions are fluid droplets.

25. The method of claim 24, wherein the fluid droplets are manipulated using digital microfluidics (DMF) mediated droplet operations.
